(19) United States
(12) Patent Application Publication
(10) Pub. No.: US 2013/0035594 A1
(43) Pub. Date: Feb. 7, 2013

(54) ULTRASONIC OBSERVATION APPARATUS, OPERATION METHOD OF THE SAME, AND COMPUTER READABLE RECORDING MEDIUM

(71) Applicant: Hirotaka EDA, Tokyo (JP)

(72) Inventor: Hirotaka EDA, Tokyo (JP)

(73) Assignee: OLYMPUS MEDICAL SYSTEMS CORP., Tokyo (JP)

(21) Appl. No.: 13/561,235

(22) Filed: Jul. 30, 2012

Related U.S. Application Data
(63) Continuation of application No. PCT/JP2011/076026, filed on Nov. 11, 2011.

(30) Foreign Application Priority Data
Nov. 11, 2010 (JP) 2010-253286

(51) Int. Cl. A61B 8/08

(52) U.S. Cl. 600/442

(57) ABSTRACT

An ultrasonic observation apparatus includes: a reference spectrum storage unit that stores a first reference spectrum in a first reception depth range and a second reference spectrum in a second reception depth range obtained based on a frequency of an ultrasonic wave received from a reference reflector; a frequency analyzer that calculates a frequency spectrum by analyzing a frequency of the received ultrasonic wave; and a corrected frequency spectrum calculator that calculates a corrected frequency spectrum by determining whether a reception depth of the frequency spectrum calculated by the frequency analyzer is the first reception depth range or the second reception depth range, and obtaining a difference, in a case of the first reception depth range, between the first reference spectrum and the frequency spectrum and a difference, in a case of the second reception depth range, between the second reference spectrum and the frequency spectrum.

### CROSS-REFERENCE TO RELATED APPLICATIONS

This application is a continuation of International Application No. PCT/JP2011/76026, designating the United States and filed on Nov. 11, 2011 which claims the benefit of priority of the prior Japanese Patent Application No. 2010-253286, filed on Nov. 11, 2010, and the entire contents of the International application and the Japanese Application are incorporated herein by reference.

### BACKGROUND OF THE INVENTION

1. Field of the Invention

The present invention relates to an ultrasonic observation apparatus that allows an observation of tissues of a subject by using ultrasonic waves, an operation method of the same, and a computer readable recording medium.

2. Description of the Related Art

Conventionally, a technique known as an ultrasound elastography has been known as a technique using ultrasonic waves for an examination of a breast cancer and the like (see International Publication No. 2005/122906, for example). The ultrasound elastography is a technique of utilizing a diagnostic that tissues developing a cancer or a tumor in an organism vary in hardness depending on a development status of a disease or on an individual. In this technique, an amount of strain and a modulus of elasticity of biological tissues in an examination site are measured by using ultrasonic waves under a condition where a compression is applied externally on the examination site and a result of the measurement is displayed as a cross-sectional image.

### SUMMARY OF THE INVENTION

According to an aspect of the present invention, an ultrasonic observation apparatus that transmits an ultrasonic wave to a subject and receives an ultrasonic wave reflected by the subject includes: a reference spectrum storage unit that stores a first reference spectrum in a first reception depth range and a second reference spectrum in a second reception depth range obtained based on a frequency of an ultrasonic wave received from a reference reflector; a frequency analyzer that calculates a frequency spectrum by analyzing a frequency of the received ultrasonic wave; and a corrected frequency spectrum calculator that calculates a corrected frequency spectrum by determining whether a reception depth of the frequency spectrum calculated by the frequency analyzer is the first reception depth range or the second reception depth range, and obtaining a difference, in a case of the first reception depth range, between the first reference spectrum and the frequency spectrum and a difference, in a case of the second reception depth range, between the second reference spectrum and the frequency spectrum.

According to another aspect of the present invention, an operation method of an ultrasonic observation apparatus that transmits an ultrasonic wave to a subject and receives an ultrasonic wave reflected by the subject includes: calculating a frequency spectrum by a frequency analyzer by analyzing a frequency of a received ultrasonic wave; storing a first reference spectrum in a first reception depth range and a second reference spectrum in a second reception depth range obtained based on a frequency of an ultrasonic wave received from a reference reflector; and calculating a corrected frequency spectrum by determining whether a reception depth of the frequency spectrum calculated at the calculating is the first reception depth range or the second reception depth range and by obtaining a difference, in a case of the first reception depth range, between the first reference spectrum and the frequency spectrum and a difference, in a case of the second reception depth range, between the second reference spectrum and the frequency spectrum.

According to still another aspect of the present invention, in a non-transitory computer readable recording medium with an executable program stored thereon, the program instructs a processor to execute: calculating a frequency spectrum by a frequency analyzer by analyzing a frequency of a received ultrasonic wave; storing a first reference spectrum in a first reception depth range and a second reference spectrum in a second reception depth range obtained based on a frequency of an ultrasonic wave received from a reference reflector; and calculating a corrected frequency spectrum by determining whether a reception depth of the frequency spectrum calculated at the calculating is the first reception depth range or the second reception depth range and by obtaining a difference, in a case of the first reception depth range, between the first reference spectrum and the frequency spectrum and a difference, in a case of the second reception depth range, between the second reference spectrum and the frequency spectrum.

The above and other features, advantages, and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram of a configuration of an ultrasonic observation apparatus according to a first embodiment of the present invention;

FIG. 2 schematically shows frequency band information stored by the ultrasonic observation apparatus according to the first embodiment of the present invention;

FIG. 3 schematically shows an outline of a creation of a reference spectrum stored by the ultrasonic observation apparatus according to the first embodiment of the present invention;

FIG. 4 is a flowchart of an outline of a process of the ultrasonic observation apparatus according to the first embodiment of the present invention;

FIG. 5 shows an example of displaying a B-mode image in a display unit of the ultrasonic observation apparatus according to the first embodiment of the present invention;

FIG. 6 is a flowchart of an outline of a process performed by a frequency analyzer of the ultrasonic observation apparatus according to the first embodiment of the present invention;

FIG. 7 schematically shows a data array of one sound ray;

FIG. 8 shows an example (first example) of a frequency spectrum calculated by the frequency analyzer of the ultrasonic observation apparatus according to the first embodiment of the present invention;

FIG. 9 shows an example (second example) of a frequency spectrum calculated by the frequency analyzer of the ultrasonic observation apparatus according to the first embodiment of the present invention;

FIG. 10 shows an example (third example) of a frequency spectrum calculated by the frequency analyzer of the ultrasonic observation apparatus according to the first embodiment of the present invention;

FIG. 11 shows an example (fourth example) of a frequency spectrum calculated by the frequency analyzer of the ultrasonic observation apparatus according to the first embodiment of the present invention;

FIG. 12 schematically shows an outline of a corrected frequency spectrum calculating process and a feature data extracting process performed on the frequency spectrum shown in FIG. 8;

FIG. 13 schematically shows an outline of a corrected frequency spectrum calculating process and a feature data extracting process performed on the frequency spectrum shown in FIG. 9;

FIG. 14 schematically shows an outline of a corrected frequency spectrum calculating process and a feature data extracting process performed on the frequency spectrum shown in FIG. 10;

FIG. 15 schematically shows an outline of a corrected frequency spectrum calculating process and a feature data extracting process performed on the frequency spectrum shown in FIG. 11;

FIG. 16 shows a new straight line defined based on feature data obtained after performing an attenuation correction on feature data related to the straight line shown in FIG. 12;

FIG. 17 is a flowchart of an outline of a process performed by a tissue property determining unit of the ultrasonic observation apparatus according to the first embodiment of the present invention;

FIG. 18 shows an example of a feature data space set by the tissue property determining unit of the ultrasonic observation apparatus according to the first embodiment of the present invention;

FIG. 19 shows an example of displaying a determination result displaying image displayed in the display unit of the ultrasonic observation apparatus according to the first embodiment of the present invention;

FIG. 20 is an explanatory view of a result of an attenuation correcting process performed by the ultrasonic observation apparatus according to the first embodiment of the present invention;

FIG. 21 is a flowchart of an outline of an attenuation correcting process performed by an ultrasonic observation apparatus according to a second embodiment of the present invention; and

FIG. 22 schematically shows the outline of the attenuation correcting process performed by the ultrasonic observation apparatus according to the second embodiment of the present invention.

### DETAILED DESCRIPTION OF THE PREFERRED EMBODIMENTS

Exemplary embodiments of the present invention (hereinafter referred to as “embodiments”) will be explained below with reference to the accompanying drawings.

### First Embodiment

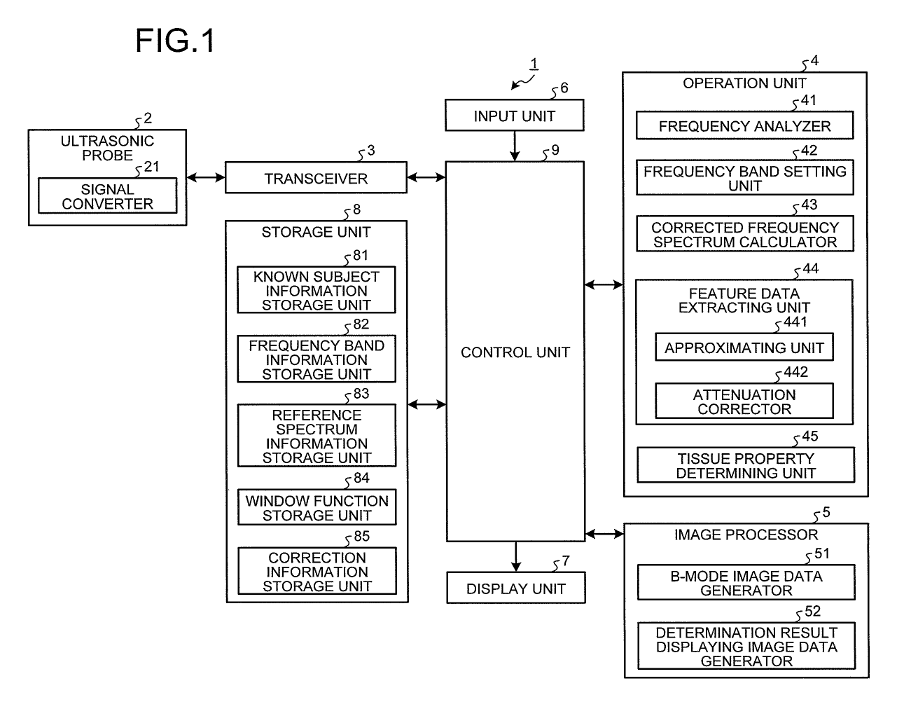

FIG. 1 is a block diagram of a configuration of an ultrasonic observation apparatus according to a first embodiment of the present invention. An ultrasonic observation apparatus 1 shown in FIG. 1 allows an observation of a subject by using ultrasonic waves.

The ultrasonic observation apparatus 1 is provided with an ultrasonic probe 2 that outputs an ultrasonic pulse to an outside and also receives an ultrasonic echo reflected at the outside, a transceiver 3 that transmits and receives an electrical signal to and from the ultrasonic probe 2, an operation unit 4 that performs a predetermined operation with respect to an electrical echo signal obtained by converting the ultrasonic echo, an image processor 5 that generates image data corresponding to the electrical echo signal obtained by converting the ultrasonic echo, an input unit 6 that is realized by using an interface such as a keyset, a mouse, and a touchscreen and accepts an input of information of various kinds, a display unit 7 that is realized by using a display panel formed by a crystal liquid or an organic EL and displays information of various kinds including images generated by the image processor 5, a storage unit 8 that stores information of various kinds including information concerning a tissue property of a known subject, and a control unit 9 that performs an operation control of the ultrasonic observation apparatus 1.

The ultrasonic probe 2 is provided with a signal converter 21 that converts the electrical pulse signal received from the transceiver 3 into an ultrasonic pulse (acoustic pulse signal) and converts the ultrasonic echo reflected by the subject outside into an electrical echo signal. The ultrasonic probe 2 may be configured such that an ultrasonic transducer mechanically scans or a plurality of ultrasonic transducers electronically scan.

The transceiver 3 is electrically connected to the ultrasonic probe 2, transmits a pulse signal to the ultrasonic probe 2, and receives an echo signal from the ultrasonic probe 2. Specifically, the transceiver 3 generates a pulse signal based on a preset waveform and transmission time and transmits the generated pulse signal to the ultrasonic probe 2. Besides, the transceiver 3 performs an A/D conversion after performing processes including amplification, filtering, and the like on the received echo signal to generate and output a digital RF signal. In the case where the ultrasonic probe 2 is configured to make a plurality of ultrasonic transducers electronically scan, the transceiver 3 includes a multichannel circuit for a beam synthesis to deal with the plurality of ultrasonic transducers.

The operation unit 4 is provided with a frequency analyzer 41 that calculates a frequency spectrum (power spectrum) of an echo signal by performing a fast Fourier transform (FFT) on the digital RF signal output from the transceiver 3, a frequency band setting unit 42 that sets a frequency band used in approximating the frequency spectrum calculated by the frequency analyzer 41, a corrected frequency spectrum calculator 43 that calculates a corrected frequency spectrum by correcting the frequency spectrum calculated by the frequency analyzer 41 based on a predetermined reference spectrum stored in the storage unit 8, a feature data extracting unit 44 that extracts feature data of a subject by performing an approximating process and an attenuation correcting process of reducing a contribution of an attenuation generated depending on a reception depth and a frequency of ultrasonic waves in the transmission of ultrasonic waves, and a tissue property determining unit 45 that determines a tissue property in a predetermined area of the subject by using the feature data extracted by the feature data extracting unit 44.

The frequency analyzer 41 calculates a frequency spectrum by performing, with respect to each sound ray (line data), the fast Fourier conversion on an FFT data group including a predetermined volume of data. A frequency spectrum shows a tendency specific to a tissue property of a subject. This is because a frequency spectrum has a correlation with size, density, acoustic impedance, and the like of a subject which is a scattering substance that scatters ultrasonic waves.

The frequency band setting unit 42 performs a frequency band setting by reading out from the storage unit 8 and referring to a frequency band table, which will be explained later, stored by the storage unit 8. The reason why the frequency band setting is changed for each reception depth in this manner is that there is a possibility in ultrasonic waves that efficient information of high frequency component is lost and inefficient information remains in an echo signal received from a site whose reception depth is large since a higher frequency component attenuates more quickly. By taking this aspect into consideration, a frequency band is set in the first embodiment so that a band width becomes narrower and a maximum frequency becomes smaller as a reception depth is larger.

The corrected frequency spectrum calculator 43 reads out from the storage unit 8 and refers to reference spectrum information, which will be explained later, stored in the storage unit 8, calculates a difference between the reference spectrum and a frequency spectrum for each reception depth, and calculate a corrected frequency spectrum. The reason why the correction of the frequency spectrum is performed for each reception depth is the same as the reason for the setting of the frequency band explained above.

The feature data extracting unit 44 is provided with an approximating unit 441 that calculates, by performing an approximating process on the corrected frequency spectrum calculated by the corrected frequency spectrum calculator 43, before-correction feature data before an attenuation correcting process is performed, and an attenuation corrector 442 that performs the attenuation correcting process on the before-correction feature data approximated by the approximating unit 441 to extract feature data.

The approximating unit 441 approximates a frequency spectrum by a primary expression via a regression analysis to extract the before-correction feature data which defines the approximate primary expression. Specifically, the feature data extracting unit 44 calculates a slope a₀ and an intercept b₀ of the primary expression via the regression analysis and also calculates intensity at a specific frequency within a frequency band in the frequency spectrum as the before-correction feature data. While the approximating unit 441 is configured to calculate an intensity (Mid-band fit) “c₀=a₀f_{MID}+b₀” in a middle frequency “f_{MID}=(f_{LOW}+f_{HIGH})/2” in the first embodiment, this is just one example. The “intensity” here indicates any one of parameters such as a voltage, an electric power, a sound pressure, and an acoustic energy.

Among the feature data of three kinds, the slope a₀ has a correlation with a size of a scattering substance that scatters ultrasonic waves and it is considered that the slope has a smaller value as the scattering substance is larger in size in general. Besides, the intercept b₀ has a correlation with a size of the scattering substance, a difference in acoustic impedance, density (consistency) of the scattering substance, and the like. Specifically, the intercept b₀ is considered to have a larger value as the scattering substance is larger in size, to have a larger value as a value for the acoustic impedance is larger, and to have a larger value as a value for the density (consistency) of the scattering substance is larger. The intensity c₀ in the middle frequency f_{MID} (hereinafter simply referred to as “intensity”) is an indirect parameter obtained from the slope a₀ and the intercept b₀ and provides spectrum intensity in the middle within an efficient frequency band. Therefore, the intensity c₀ is considered to have a certain level of correlation with a brightness of the B-mode image in addition to the size of the scattering substance, the difference in acoustic impedance, and the density of the scattering substance. Here, an approximating polynomial calculated by the feature data extracting unit 44 is not limited to the primary expression and an approximating polynomial of quadratic or higher expression may be used.

A correction performed by the attenuation corrector 442 will be explained. An attenuation amount A of ultrasonic waves can be expressed as follows:

A=2αzf (1)

Here, a symbol “α” indicates an attenuation rate, a symbol “z” indicates a reception depth of ultrasonic waves, and a symbol “f” indicates a frequency. As evidenced by expression (1), the attenuation amount A is proportional to the frequency f. A specific value for the attenuation rate α is 0 to 1.0 (dB/cm/MHz), more preferably 0.3 to 0.7 (dB/cm/MHz) in a case of a biological body, and the value is determined depending on the kind of an organ as an observation target. For example, in a case where an organ as an observation target is a pancreas, “α=0.6 (dB/cm/MHz)” is determined. Here in the first embodiment, it is also possible to make a configuration such that the value for the attenuation rate a can be changed by an input from the input unit 6.

The attenuation corrector 442 corrects the before-correction feature data (the slope a₀, the intercept b₀, and the intensity c₀) extracted by the approximating unit 441 as follows.

a=a₀+2αz (2)

b=b₀ (3)

c=c₀+2αzf_{MID} (=af_{MID}+b) (4)

As evidenced by expressions (2) and (4), the attenuation corrector 422 performs a correction whose correction amount is larger as the reception depth z of ultrasonic waves is larger. Besides, according to expression (3), a correction concerning to the intercept is an identical transformation. This is because the intercept is a frequency component corresponding to the frequency 0 (Hz) and is not subject to the attenuation.

The tissue property determining unit 45 calculates an average and a standard deviation of feature data of the frequency spectrum extracted by the feature data extracting unit 44 for each feature data. The tissue property determining unit 45 determines a tissue property of a predetermined area of the subject by using the calculated average and the standard deviation and an average and a standard deviation of feature data, stored in the storage unit 8, of a frequency spectrum of a known subject. The “predetermined area” here means an area in an image specified, via the input unit 6, by an operator of the ultrasonic observation apparatus 1 who watches images generated by the image processor 5 (hereinafter referred to as “area of interest”). Besides, the “the tissue property” here means any one of a cancer, an endocrine tumor, a mucinous tumor, normal tissues, and a vascular channel, for example. In the case where the subject is a pancreas, a chronic pancreatitis, an autoimmune pancreatitis, and the like are included as the tissue property.

The average and the standard deviation of the feature data calculated by the tissue property determining unit 45 reflect changes at a cellular level such as an enlargement of a nucleus and a heteromorphy and changes in tissues such as a fibrous growth in interstitium and a fibrosis substituted with parenchymal tissues, and indicate a value specific to each tissue property. Therefore, it becomes possible to accurately determine a tissue property in a predetermined area of the subject by using the average and the standard deviation of the feature data.

The image processor 5 is provided with a B-mode image data generator 51 that generates B-mode image data for performing a display by converting an amplitude of an echo signal into a brightness and a determination result displaying image data generator 52 that generates a determination result displaying image data for performing a display of a determination result of the tissue property in the area of interest and information related to the determination result by using the data output by the B-mode image data generator 51 and the operation unit 4.

The B-mode image data generator 51 generates B-mode image data by performing a signal process using known techniques such as a band-pass filter, a logarithmic transformation, a gain process, and a contrast process on the digital signal, and also culling data depending on a data step width which is determined in accordance with a display range of an image in the display unit 7.

The determination result displaying image data generator 52 generates determination result displaying image data including the determination result of the tissue property in the area of interest and a tissue property emphasized image in which the tissue property is emphasized by using the B-mode image data generated by the B-mode image data generator 51, the feature data extracted by the feature data extracting unit 44, and the determination result determined by the tissue property determining unit 45.

The storage unit 8 is provided with a known subject information storage unit 81 that stores information of a known subject, a frequency band information storage unit 82 that stores frequency band information determined depending on a reception depth of ultrasonic waves, a reference spectrum information storage unit 83 that stores reference spectrum information depending on a reception depth of ultrasonic waves, a window function storage unit 84 that stores a window function which is used in a frequency analyzing process performed by the frequency analyzer 41, and a correction information storage unit 85 that stores correction information which is referred to when an attenuation corrector 442 performs the process.

The known subject information storage unit 81 stores, by associating with a tissue property of a known subject, feature data of a frequency spectrum extracted with respect to the known subject. Besides, the known subject information storage unit 81 stores, with respect to the feature data of the frequency spectrum related to the known subject, an average and a standard deviation calculated for each of groups classified based on tissue properties of known subjects, together with the data of all kinds of the feature data of the known subjects. Here, the feature data of the known subjects is extracted in the same process as the first embodiment. It should be noted that it is not necessary to perform the process of extracting feature data of the known subjects in the ultrasonic observation apparatus 1. It is preferable that information of the known subjects stored in the known subject information storage unit 81 has a high degree of reliability on tissue property.

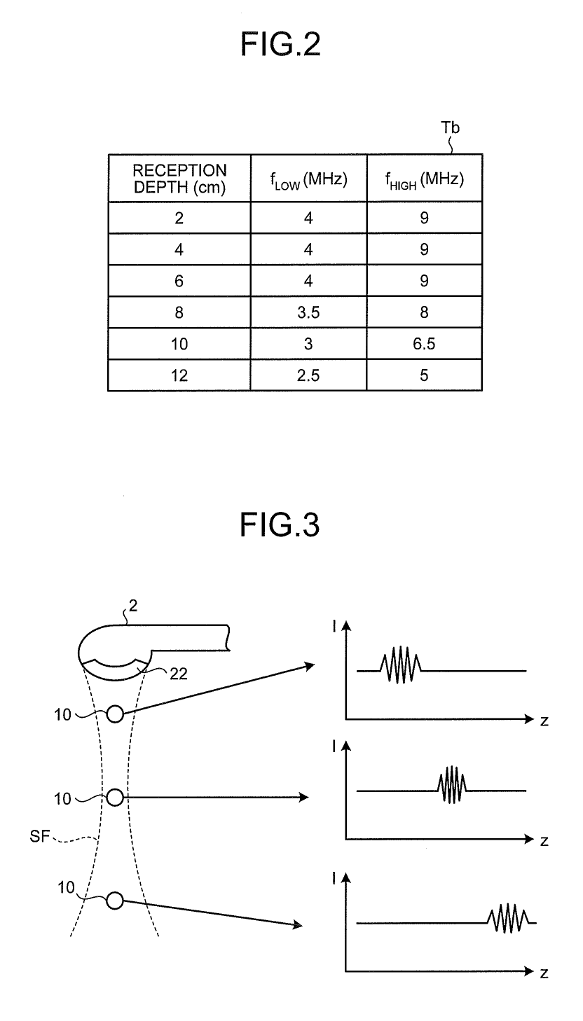

FIG. 2 schematically shows a frequency band table as frequency band information stored in the frequency band information storage unit 82. A frequency band table Tb in FIG. 2 shows a minimum frequency (f_{LOW}) and a maximum frequency (f_{HIGH}) for each reception depth of ultrasonic waves. In the frequency band table Tb, the larger the reception depth is, the narrower a band width F_{HIGH}−F_{LOW} is and the smaller the maximum frequency f_{HIGH} is. Besides, when the reception depth is relatively small (2 to 6 cm in FIG. 2), the frequency band is not changed in the frequency band table Tb since an influence of an attenuation is small. In contrast, when the reception depth is relatively large (8 to 12 cm in FIG. 2), the band is made narrow and made to shift to a side of a lower frequency since an influence of an attenuation is large. By using the frequency band table Tb, it is possible to perform imaging by extracting only a signal having efficient information. Here, the frequency band table is set individually for each kind (model) of the ultrasonic probe 2.

The reference spectrum information storage unit 83 stores, as frequency information depending on each reception depth of ultrasonic waves on a predetermined reference reflector, a frequency spectrum calculated based on an echo signal obtained by being reflected by the reference reflector (hereinafter referred to as “reference spectrum”). The reference reflector is an ideal reflector on which ultrasonic waves do not scatter, through which ultrasonic waves do not pass, and by which ultrasonic waves are not absorbed, for example. The reference spectrum is calculated for each kind of the ultrasonic probe 2 and for each reception depth of ultrasonic waves. Here, the reason why the reference spectrum is calculated for each of different ultrasonic probes 2 is that a transducer differs depending on the kind of the ultrasonic probes 2 and therefore there is a difference in a waveform of pulse to be transmitted. Here, it is not necessary that the reference reflector is the ideal reflector in a sense explained above.

FIG. 3 schematically shows an outline of a process of creating the reference spectrum. As shown in FIG. 3, a transducer 22 provided in the ultrasonic probe 2 forms a sound field (SF) nearly symmetric with respect to a travelling direction (vertical direction in FIG. 3) of ultrasonic waves around a focal point. FIG. 3 illustrates a relation between a reception depth z and an intensity I of each echo signal obtained by the ultrasonic probe 2 when a reference reflector 10 is arranged at three points including the focal point. A reference spectrum is calculated via a frequency analysis by the frequency analyzer 41 by using intensity data of an echo signal reflected by the reference reflector 10 in calculating the reference spectrum, and a result of the calculation is stored in the reference spectrum information storage unit 83.

The window function storage unit 84 stores at least one of window functions such as Hamming window, Hanning window, and Blackman window.

The correction information storage unit 85 stores information concerning conversion of expressions (2) to (4).

The storage unit 8 is realized by using a ROM that stores in advance an operation program of the ultrasonic observation apparatus according to the first embodiment, a program for starting the operation system, and the like, and a RAM that stores operation parameters of various processes, data, and the like.

Components other than the ultrasonic probe 2 of the ultrasonic observation apparatus 1 having functional configuration explained above are realized by using a computer provided with a CPU having an operating function and a controlling function. The CPU provided in the ultrasonic observation apparatus 1 executes an operating process related to an operating method of the ultrasonic observation apparatus according to the first embodiment by reading out, from the storage unit 8, information memorized and stored in the storage unit 8 and programs of various kinds including the operation program of the ultrasonic observation apparatus explained above.

Here, it is possible to widely distribute the operation program of the ultrasonic observation apparatus according to the first embodiment by recording it in a computer-readable recording medium such as a hard disk, a flash memory, a CD-ROM, a DVD-ROM, and a flexible disk.

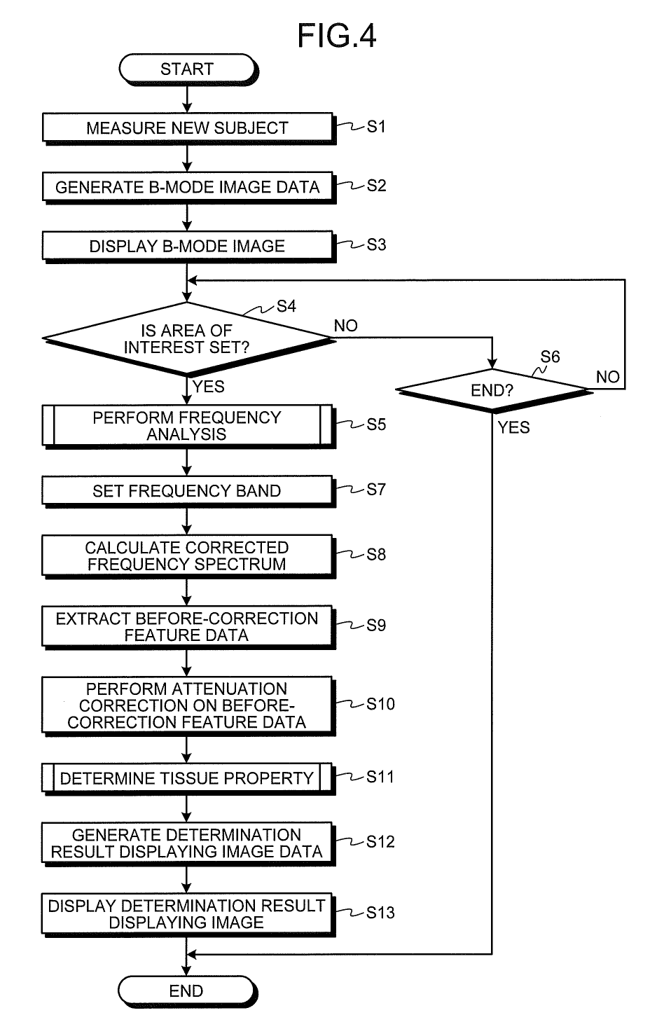

FIG. 4 is a flowchart of an outline of a process of the ultrasonic observation apparatus 1 having the configuration explained above. In FIG. 4, the ultrasonic observation apparatus 1 first performs a measurement of a new subject by the ultrasonic probe 2 (step S1). After that, the B-mode image data generator 51 generates B-mode image data (step S2).

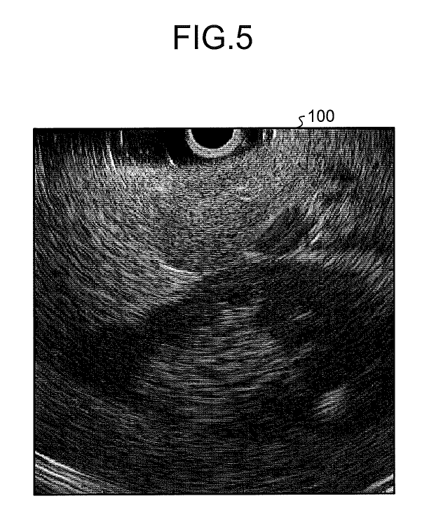

The control unit 9 then performs a control of making the display unit 7 display a B-mode image corresponding to the B-mode image data generated by the B-mode image data generator 51 (step S3). FIG. 5 shows an example of displaying a B-mode image in the display unit 7. A B-mode image 100 shown in FIG. 5 is a gray-scale image in which values for variables R (red), G (green), and B (blue), when an RGB color system is adopted for a color space, are made to conform.

After that, when an area of interest is set via the input unit 6 (“Yes” at step S4), the frequency analyzer 41 calculates a frequency spectrum by performing a frequency analysis through the FFT operation (step S5). At this step S5, it is possible to set all area of the image as the area of interest. On the other hand, when an area of interest is not set (“No” at step S4) and an instruction to end the process is input via the input unit 6 (“Yes” at step S6), the ultrasonic observation apparatus 1 ends the process. In contrast, when an area of interest is not set (“No” at step S4) and the instruction to end the process is not input (“No” at step S6), the ultrasonic observation apparatus 1 returns to step S4.

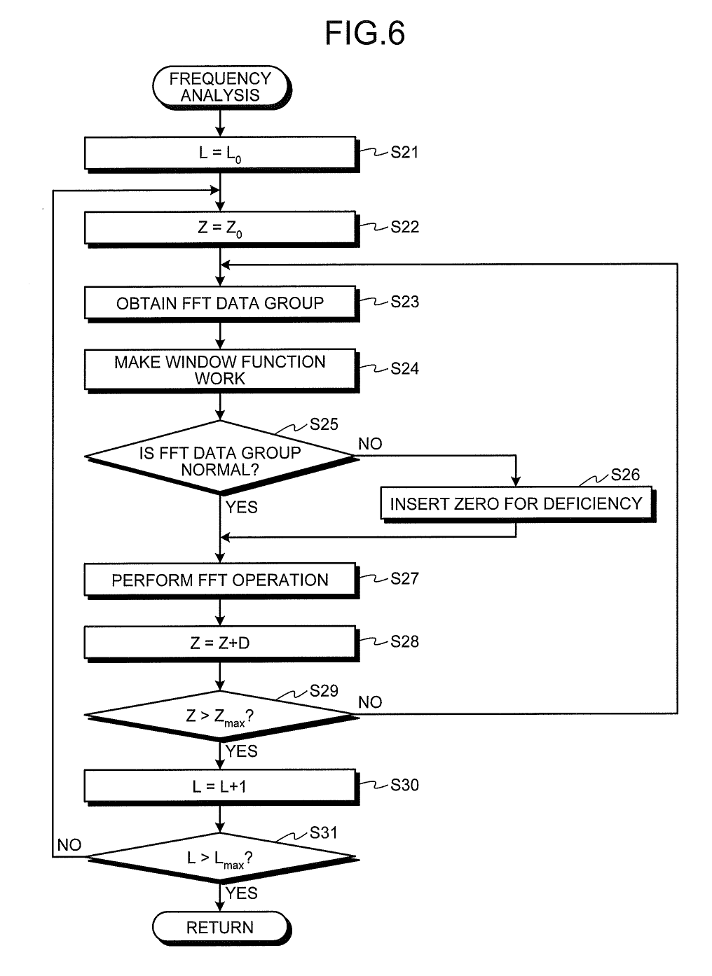

Here, the process (step S5) performed by the frequency analyzer 41 will be explained in detail with reference to the flowchart shown in FIG. 6. The frequency analyzer 41 first sets a sound ray number L of a sound ray as the first analysis target to an initial value L₀ (step S21). The initial value L₀ may be provided to a sound ray that the transceiver 3 receives for the first time, or to a sound ray corresponding to a border position at one of the left and the right of the area of interest set via the input unit 6.

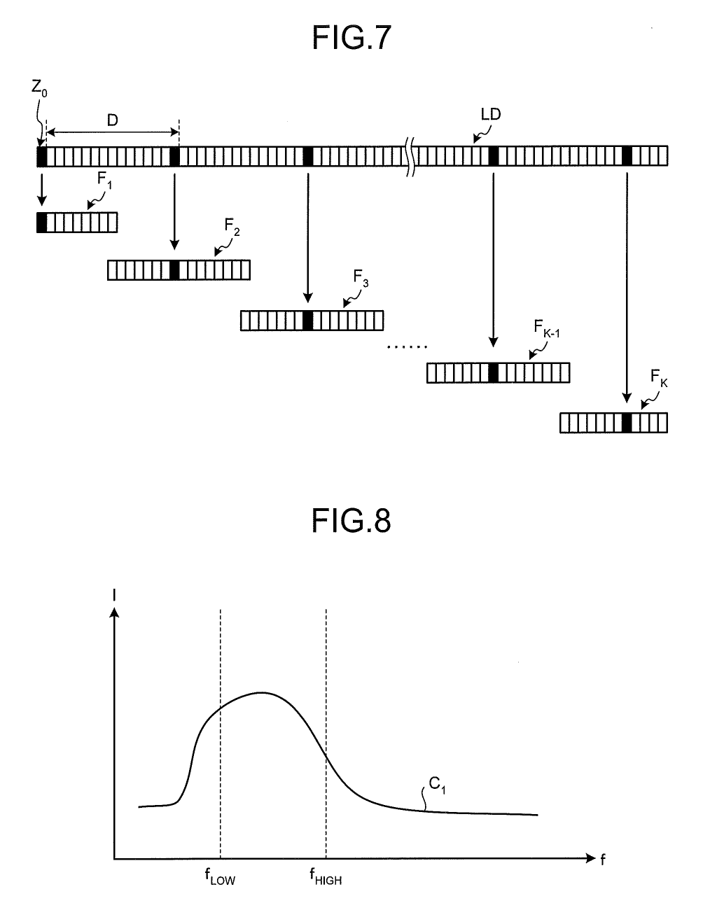

The frequency analyzer 41 then calculates all frequency spectra for a plurality of data positions set on one sound ray. The frequency analyzer 41 first sets an initial value Z₀ for a data position Z (corresponding to a reception depth) which represents a series of data group (FFT data group) obtained for the FFT operation (step S22). FIG. 7 schematically shows a data array of one sound ray. In the sound ray LD shown in FIG. 7, a white or a black rectangle means one piece of data. The sound ray LD is discretized by a time interval corresponding to a sampling frequency (50 MHz, for example) in the A/D conversion performed by the transceiver 3. FIG. 7 shows a case where first data piece in the sound ray A/D is set to the initial value Z₀ for the data position Z. Here, FIG. 7 shows merely one example and a position of the initial value Z₀ may be arbitrarily set. For example, data position Z corresponding to an upper end position of the area of interest may be set to the initial value Z₀.

After that, the frequency analyzer 41 obtains FFT data group of the data position Z (step S23) and makes the window function stored in the window function storage unit 84 work on the obtained FFT data group (step S24). By making the window function work on the FFT data group, it is possible to avoid a discontinuity of the FFT data group at a border and prevent an occurrence of an artifact.

The frequency analyzer 41 then determines whether or not the FFT data group of the data position Z is a normal data group (step S25). Here, it is necessary that the FFT data group has data pieces whose number is a power of two. The number of data pieces of the FFT data group will be expressed as 2ⁿ (“n” being a positive integer) below. The description “the FFT data group is normal” means that the data position Z locates at a 2ⁿ⁻¹th position from the front in the FFT data group. In other words, the description “the FFT data group is normal” means that there is 2ⁿ⁻¹−1 (=N) pieces of data before the data position Z and there is 2ⁿ⁻¹ (=M) pieces of data after the data position Z. In the case shown in FIG. 7, while FFT data groups F₂, F₃, and F_{K-1} are normal, FFT data groups F₁ and F_{K} are abnormal. In FIG. 7, the positive integer “n” is 4 (N=7, M=8).

As a result of the determination at step S25, when the FFT data group of the data position Z is normal (“Yes” at step S25), the frequency analyzer 41 moves to step S27, which will be explained later.

As a result of the determination at step S25, when the FFT data group of the data position Z is not normal (“No” at step S25), the frequency analyzer 41 generates a normal FFT data group by inserting zero for the deficiency (step S26). The FFT data group determined not to be normal at step S25 is worked on by the window function before the insertion of zero. Therefore, no discontinuity of data occurs even by inserting zero to the FFT data group. After step S26, the frequency analyzer 41 moves to step S27, which will be explained later.

At step S27, the frequency analyzer 41 obtains a frequency spectrum by performing the FFT operation by using the FFT data group (step S27).

The frequency analyzer 41 then adds a predetermined data step width D to the data position Z and calculates a data position Z of an FFT data group as a next analysis target (step S28). While it is preferable that the data step width D here is made to accord with the data step width used when the B-mode image data generator 51 generates the B-mode image data, a value larger than the data step width used by the B-mode image data generator may be set if it is requested to reduce an operation amount in the frequency analyzer 41. FIG. 7 shows a case where the data step width D is 15.

After that, the frequency analyzer 41 determines whether or not the data position Z is larger than a last data position Zₘₐₓ (step S29). Here, the last data position Zₘₐₓ may be configured to be a data length of the sound ray LD or to be a data position corresponding to a lower end of the area of interest. When the data position Z is larger than the last data position Zₘₐₓ as a result of the determination (“Yes” at step S29), the frequency analyzer 41 increases the sound ray number L by one (step S30). On the other hand, when the data position Z is not larger than the last data position Zₘₐₓ (“No” at step S29), the frequency analyzer 41 returns to step S23. In this manner, the frequency analyzer 41 performs the FFT operation on FFT data groups whose number is [{(Zₘₐₓ−Z₀)/D}+1] (=K) with respect to one sound ray LD. Here, an integer [X]indicates a maximum integer not exceeding X.

When the sound ray number L after the increase at step S30 is larger than a last sound ray number Lₘₐₓ (“Yes” at step S31), the frequency analyzer 41 returns to the main routine shown in FIG. 2. On the other hand, when the sound ray number L after the increase at step S30 is not more than the last sound ray number Lₘₐₓ (“No” at step S31), the frequency analyzer 41 returns to step S22.

In this manner, the frequency analyzer 41 performs the FFT operation K times with respect to each of (Lₘₐₓ−L₀+1) sound rays. Here, the last sound ray number Lₘₐₓ may be provided to the last sound ray that the transceiver 3 receives, or to a sound ray corresponding to a border at one of the left and the right of the area of interest, for example. A total number (Lₘₐₓ−L₀+1)×K of the FFT operations performed by the frequency analyzer 41 with respect to all the sound rays will be treated as “P” below.

After the frequency analyzing process at step S5 explained above, the frequency band setting unit 42 performs a frequency band setting for each reception depth of ultrasonic waves with reference to the frequency band table Tb stored in the frequency band information storage unit 82 (step S7). Here, the process of the frequency band setting unit 42 may be performed in parallel with the process of the frequency analyzer 41 or may be performed prior to the process of the frequency analyzer 41.

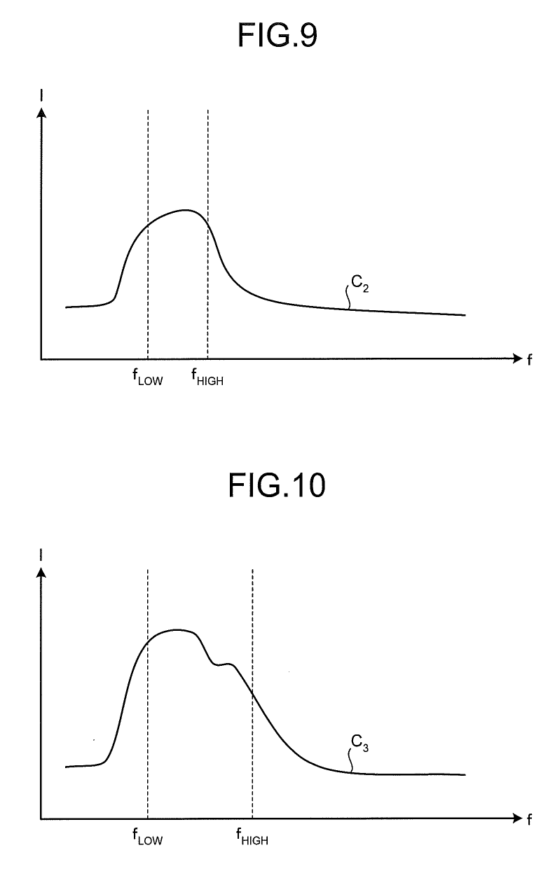

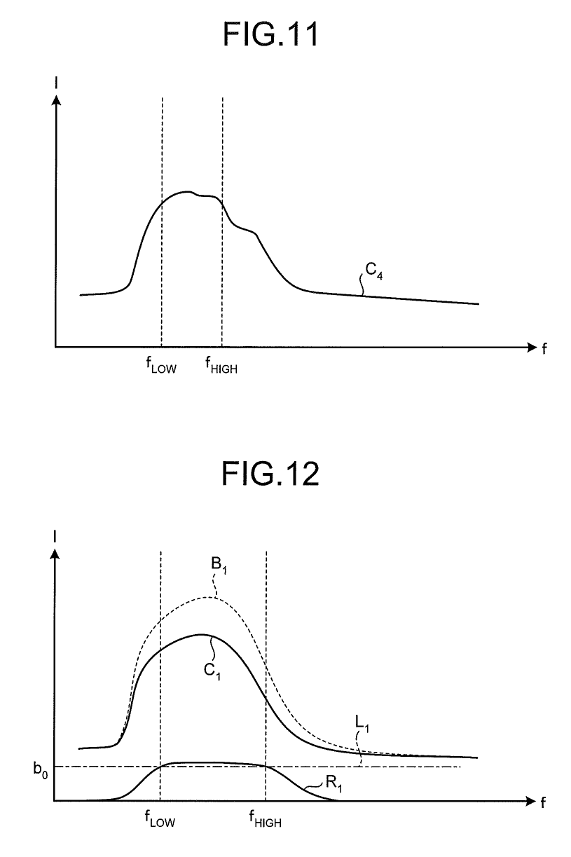

FIGS. 8 to 11 schematically show frequency spectra calculated by the frequency analyzer 41 and frequency bands set by the frequency band setting unit 42 with respect to the frequency spectra, respectively. FIGS. 8 to 11 show four kinds of frequency spectra and frequency bands with respect to a subject having the same tissue property, each one of FIGS. 8 to 11 being different from the others in at least one of the reception depth and the ultrasonic probe 2. In the figures, spectrum curves C₁ and C₂ respectively shown in FIGS. 8 and 9 show frequency spectra in respectively different reception depths when the same ultrasonic probe 2 is used. Here, the reception depth corresponding to the spectrum curve C₁ is smaller than the reception depth corresponding to the spectrum curve C₂. Besides, spectrum curves C₃ and C₄ respectively shown in FIGS. 10 and 11 show frequency spectra in respectively different reception depths when the same ultrasonic probe 2 which is, however, different from the ultrasonic probe 2 used in obtaining the spectrum curves C₁ and C₂ is used. Here, the reception depth corresponding to the spectrum curve C₃ is smaller than the reception depth corresponding to the spectrum curve C₄. Here, a curve and a straight line as a frequency function are formed by an aggregate of discrete dots in the first embodiment. In this respect, the same applies to embodiments, which will be explained later.

The reception depth corresponding to the spectrum curve C₁ and the reception depth corresponding to the spectrum curve C₃ are the same. Besides, the reception depth corresponding to the spectrum curve C₂ and the reception depth corresponding to the spectrum curve C₄ are the same. Frequency band is defined depending on the kind of the ultrasonic probe 2. As explained above, the reception depth in the case shown in FIGS. 9 and 11 is larger than the case shown in FIGS. 8 and 10. Therefore, a band width f_{HIGH}-f_{LOW} of a frequency band in the case shown in FIGS. 9 and 11 is narrower than the case shown in FIGS. 8 and 10.

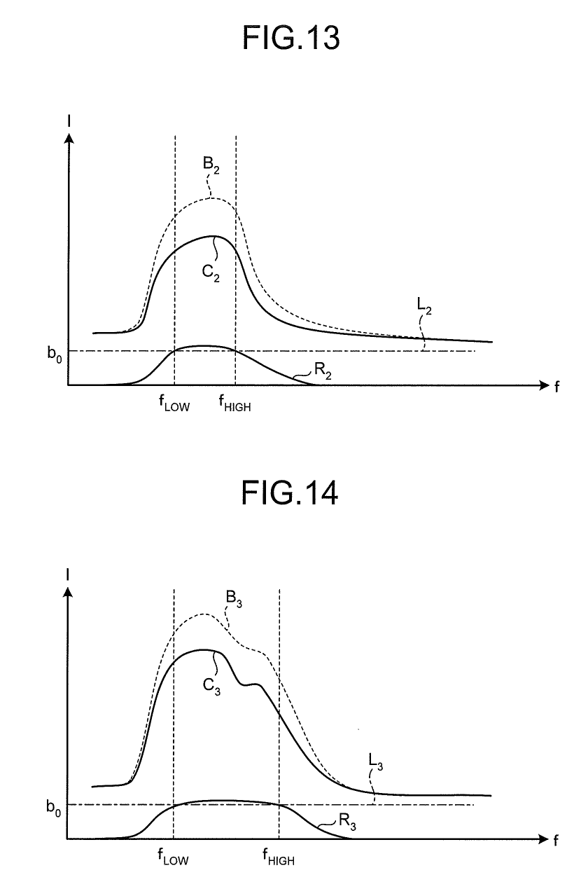

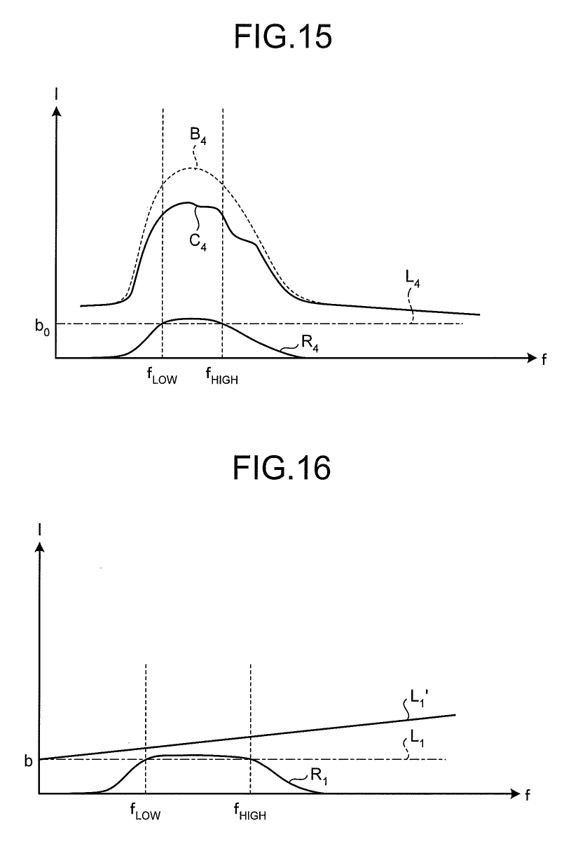

The corrected frequency spectrum calculator 43 then reads out from the reference spectrum information storage unit 83 and refers to a reference spectrum depending on the reception depth and the kind of the ultrasonic probe 2, and calculates a difference between the reference spectrum and the frequency spectrum calculated by the frequency analyzer 41 to calculate a corrected frequency spectrum (step S8.) FIGS. 12 to 15 schematically show outlines of corrected frequency spectrum calculating processes for the spectrum curves C₁ to C₄, respectively. Curves B₁ to B₄ respectively shown in FIGS. 12 to 15 show reference spectrum curves depending on the reception depth and the kind of the ultrasonic probe 2. The corrected frequency spectrum calculator 43 calculates corrected frequency spectrum curves R₁ to R₄ by taking absolute values of differences between the reference spectrum curves B₁ to B₄ and the frequency spectrum curves C₁ to C₄, respectively. Straight lines L₁ to L₄ respectively shown in FIGS. 12 to 15 will be explained in a feature data extracting process, which will be explained later.

After step S8, the approximating unit 441 extracts before-correction feature data via the regression analysis on the frequency spectra whose number is P calculated by the frequency analyzer 41 as an approximating process (step S9). Specifically, the approximating unit 441 extracts, by calculating a primary expression that approximates a frequency spectrum of a frequency band f_{Low}<f<f<f_{HIGH} via the regression analysis, the slope a₀, the intercept b₀, and the intensity c₀ which define the primary expression as before-correction feature data. Straight lines L₁ to L₄ respectively shown in FIGS. 12 to 15 are regression lines obtained by performing the regression analysis on the frequency spectrum curves C₁ to C₄, respectively at step S9. In the first embodiment, the setting of frequency band and the calculation of corrected frequency spectrum are performed prior to the extraction of feature data. Therefore, the straight lines L₁ to L₄ are just the same straight lines. In other words, feature data having the same value is extracted irrespective of the reception depth and the kind of the ultrasonic probe 2 according to the first embodiment.

After this, the attenuation corrector 442 performs the attenuation correcting process on the before-correction feature dada extracted by the approximating unit 441 (step S10). In a case where a data sampling frequency is 50 MHz, for example, a time interval of the data sampling is 20 (nsec). Here, assuming that a sound velocity is 1530 (m/sec), an interval in distance of the data sampling is “1530 (m/sec)×20 (nsec)/2=0.0153 (mm)”. Assuming that the number of data steps from the first data piece in the sound ray LD to a data position in an FFT data group as a processing target is k, the data position Z becomes 0.0153 k (mm). The attenuation corrector 442 calculates the slop a, the intercept b, and the intensity c, which are the feature data of the frequency spectrum, by substituting the value for the data position Z obtained in this manner in the reception depth z in expressions (2) to (4) explained above. FIG. 16 shows a straight line defined based on the feature data obtained after performing the attenuation correction on feature data related to the straight line L₁ shown in FIG. 12. An expression for a line L₁′ shown in FIG. 16 is as follows.

I=af+b=(a₀+2αZ)f+b₀ (5)

As evidenced by expression (5), the straight line L₁′ has a slop whose inclination is large and has the same value in intercept, compared to the straight line L₁.

After this, the tissue property determining unit 45 determines a tissue property in the area of interest of the subject based on the feature data extracted by the feature data extracting unit 44 and the known subject information stored in the known subject information storage unit 81 (step S11).

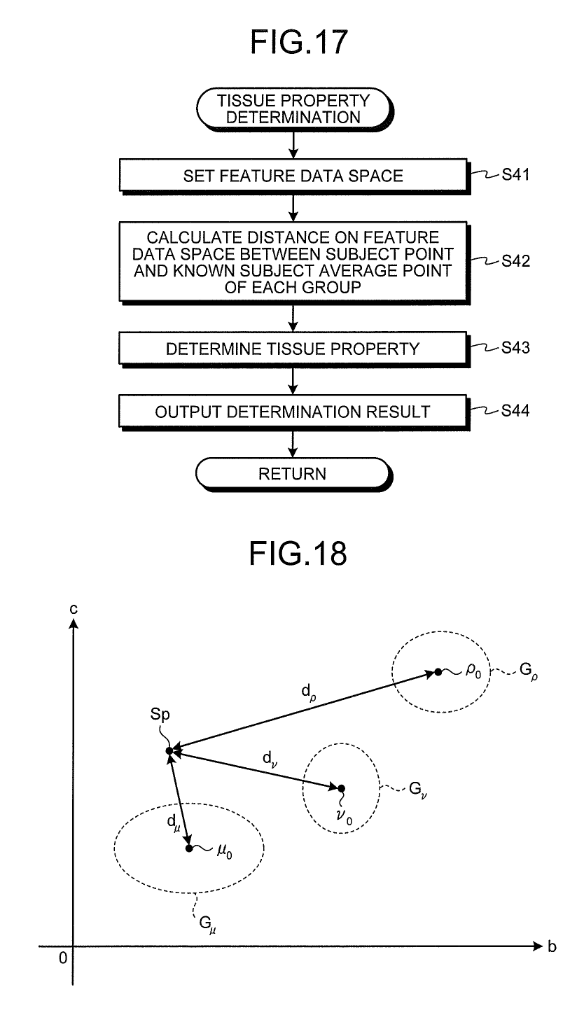

Here, the process (step S11) performed by the tissue property determining unit 45 will be explained in detail with reference to the flowchart shown in FIG. 17. The tissue property determining unit 45 first sets a feature data space used in determining a tissue property (step S41). In the first embodiment, independent parameters are two in the feature data of the three kinds, i.e., the slope a, the intercept b, and the intensity c. Therefore, it is possible to set a two-dimensional space whose components are given two kinds among the three kinds of the feature data as the feature data space. It is also possible to set one-dimensional space whose component is one kind among the three kinds of the feature data as the feature data space. While the feature data space to set is assumed to be determined in advance at step S41, a desired feature data space may be selected by the operator via the input unit 6.

FIG. 18 shows an example of the feature data space set by the tissue property determining unit 45. In the feature data space shown in FIG. 18, the horizontal axis indicates the intercept b and the vertical axis indicates the intensity c. A dot Sp shown in FIG. 18 indicates a point having, as a coordinate in the feature data space, the intercept b and the intensity c which are calculated with respect to the subject as a determination target (hereinafter referred to as “subject point”). Besides, areas G_{μ}, G_{ν}, and G_{ρ} shown in FIG. 18 indicate that respective tissue properties of the known subjects stored in the known subject information storage unit 81 are μ, ν, and ρ, respectively. In the case shown in FIG. 18, the three groups G_{μ}, G_{ν}, and G_{ρ} are present in respective areas each of which is isolated from other groups in the feature data space.

Since the classification and the determination of tissue properties also in obtaining feature data of a known subject are performed by using, as an indicator, feature data obtained via the attenuation correction on before-correction feature data of frequency spectrum obtained by the frequency analysis in the first embodiment, it is possible to clearly distinguish tissue properties which differ from each other. Especially, since feature data on which the attenuation correction is performed is used in the first embodiment, it is possible to obtain an area of each group in the feature data space in a condition where groups are separated more clearly, compared to the case of using feature data extracted without performing the attenuation correction.

After step S41, the tissue property determining unit 45 calculates distances d_{μ}, d_{ν}, and d_{ρ}, on the feature data space, between the subject point Sp and respective points μ₀, ν₀, and ρ₀ each of which has, as a coordinate in the feature data space, an average of intercepts b and an average of intensities c of frequency spectra in FFT data groups included in each of the groups G_{μ}, G_{ν}, and G_{ρ}, the points being hereinafter referred to as “known subject average point” (step S42). Here, when b-axis component and c-axis component in the feature data space differ significantly in scale, it is preferable to arbitrarily perform a weighting so that contributions of respective distances become nearly uniform.

The tissue property determining unit 45 then determines a tissue property of all the subject points including the subject point Sp based on the distances calculated at step S42 (step S43). For example, since the distance d_{μ} is the smallest in the case shown in FIG. 18, the tissue property determining unit 45 determines that the tissue property of the subject should be μ. When the subject point Sp is separated away from the known subject average points μ₀, ν₀, and ρ₀ extremely, a degree of reliability for the determination result on the tissue property is low even if the smallest value among the distances d_{μ}, d_{ν}, and d_{ρ} is obtained. So, when the distances d_{μ}, d_{ν}, and d_{ρ} are larger than a predetermined threshold value, the tissue property determining unit 45 may output an error signal. Besides, when there arise two or more smallest values among the distances d_{μ}, d_{ν}, and d_{ρ}, the tissue property determining unit 45 may select all tissue properties corresponding to the smallest values each as a candidate or select any one of the tissue properties in accordance with a predetermined rule. In the latter situation, a method of placing a high priority on a tissue property whose degree of malignancy is high like a cancer can be listed. Besides, when there arise two or more smallest values among the distances d_{μ}, d_{ν}, and d_{ρ}, the tissue property determining unit 45 may output an error signal.

After this, the tissue property determining unit 45 outputs the result of the distance calculation at step S42 and the result of the determination at step S43 (step S44). Thus, the tissue property determining process at step S11 is ended.

After step S11 explained above, the determination result displaying image data generator 52 generates determination result displaying image data by using the B-mode image data generated by the B-mode image data generator 51, the feature data calculated by the feature data extracting unit 44, and the determination result determined by the tissue property determining unit 45 (step S12).

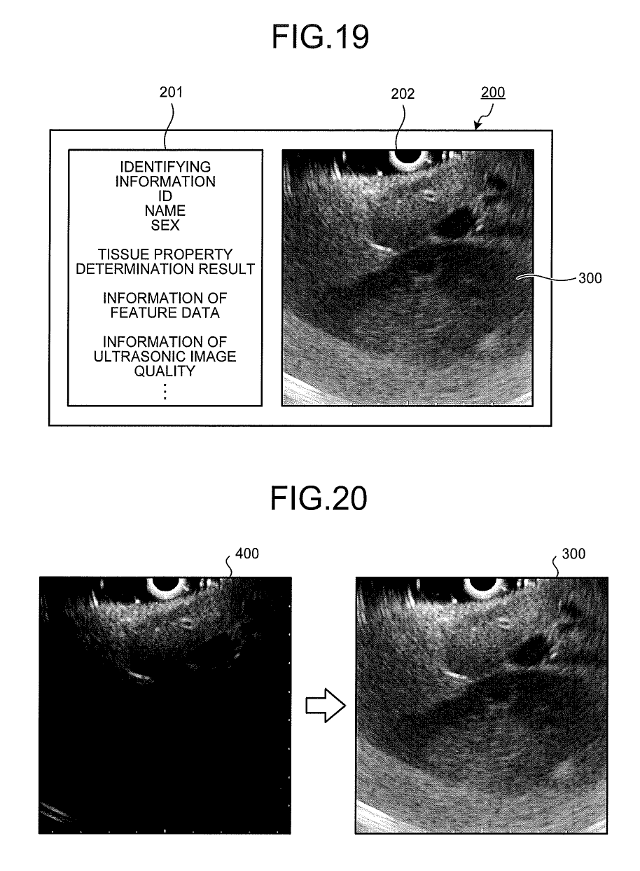

The display unit 7 then displays the determination result displaying image generated by the determination result displaying image data generator 52 (step S13). FIG. 19 shows an example of displaying a determination result displaying image displayed in the display unit 7. A determination result displaying image 200 shown in FIG. 19 includes an information displaying part 201 that displays related information of various kinds including the determination result on the tissue property and an image displaying part 202 that displays a tissue property emphasized image in which the tissue property is emphasized based on the B-mode image.

In the information displaying part 201, identifying information (ID number, name, sex, and the like) of the subject for example, the tissue property determination result obtained by the tissue property determining unit 45, information concerning the feature data in performing the tissue property determination, and information of ultrasonic image quality such as a gain and a contrast are displayed. Here, for the information concerning the feature data, it is possible to make a display utilizing an average and a standard deviation of feature data of frequency spectra of FFT data groups, the number of which is Q, present in an inside of the area of interest.

Specifically, it is possible to display “Slope=1.5±0.3 (dB/MHz), Intercept=−60±2 (dB), and Intensity=−50±1.5 (dB)”, for example in the information displaying part 201.

A tissue property emphasized image 300 displayed in the image displaying part 202 is a gray-scale image in which the intercept b is uniformly allotted to R (red), G (green), and B (blue) with respect to the B-mode image 100 shown in FIG. 5.

Due to the display of the determination result displaying image 200 having the configuration explained above by the display unit 7, it becomes possible for the operator to grasp the tissue property in the area of interest more accurately. Here, the determination result displaying image is not limited to the configuration explained above. For example, the tissue property emphasized image and the B-mode image may be displayed side by side for the determination result displaying image. Thus, it is possible to recognize the difference between the two images on one frame.

FIG. 20 is an explanatory view of a result of the attenuation correcting process performed by the ultrasonic observation apparatus 1. An image 400 shown in FIG. 20 is a tissue property emphasized image in the case where the attenuation correction is not performed. In the tissue property emphasized image 400, a signal intensity becomes lowered due to an influence of the attenuation in an area whose reception depth is large (downward area in FIG. 20) and an image becomes dark. In contrast, it is apparent that an image whose brightness is uniform over the entirety of the frame is obtained in the tissue property emphasized image 300 on which the attenuation correction is performed.

The tissue property emphasized image 300 shown in FIGS. 19 and 20 is just one example. In addition, it is possible to display the tissue property emphasized image in a color image by allotting the slope a, the intercept b, and the intensity c respectively to R (red), G (green), and B (blue), for example. In this case, since a tissue property is expressed by a specific color, it is possible for the operator to grasp the tissue property in the area of interest based on the color distribution of the image. Besides, instead of constituting a color space in the RGB color system, a color space may be constituted by variables for complementary colors such as cyan, magenta, and yellow and feature data may be allotted to respective variables. Besides, tissue property emphasized image data may be generated by mixing, by a predetermined ratio, the B-mode image data and color image data. Besides, tissue property emphasized image data may be generated by making only the area of interest replaced with color image data.

According to the first embodiment explained so far, it is possible to clearly determine the difference in tissues without using an amount of strain and a modulus of elasticity of biological tissues since a frequency spectrum is calculated by analyzing a frequency of received ultrasonic waves, a frequency band used in approximating the frequency spectrum is set, the frequency spectrum is corrected based on a reference spectrum read out from the storage unit that stores the reference spectrum obtained based on a frequency of ultrasonic waves received from the reference reflector, before-correction feature data is extracted by performing the approximating process on the corrected frequency spectrum, and then feature data of the subject is extracted by performing the attenuation correcting process in which the contribution of ultrasonic attenuation which depends on the reception depth and the frequency of ultrasonic waves is reduced. Hence, it is possible to enable distinguishing a tissue property accurately and to enhance the reliability for the observation result.

According to the first embodiment, it is possible to remove an influence of the attenuation associated with the transmission of ultrasonic waves and to perform a tissue property determination with higher accuracy since the attenuation correction is performed on the extracted feature data.

According to the first embodiment, it is possible to remove an influence of the attenuation associated with the transmission of ultrasonic waves and to perform a tissue property determination with even higher accuracy since the frequency band is determined so that the band width becomes narrower and the maximum frequency becomes smaller as the reception depth is larger.

### Second Embodiment

A second embodiment of the present invention differs from the first embodiment in the feature data extracting process performed by the feature data extracting unit. A configuration of an ultrasonic observation apparatus according to the second embodiment is the same as that of the ultrasonic observation apparatus 1 explained in the first embodiment. Therefore, an identical component corresponding to a component of the ultrasonic observation apparatus 1 will be assigned with the same reference symbol in the explanation below.

In a feature data extracting process according to the second embodiment, the attenuation corrector 442 first performs the attenuation correcting process on the corrected frequency spectrum calculated by the corrected frequency spectrum calculator 43. After that, the approximating unit 441 extracts feature data of the frequency spectrum by performing the approximating process on the corrected frequency spectrum on which the attenuation correction is performed by the attenuation corrector 442.

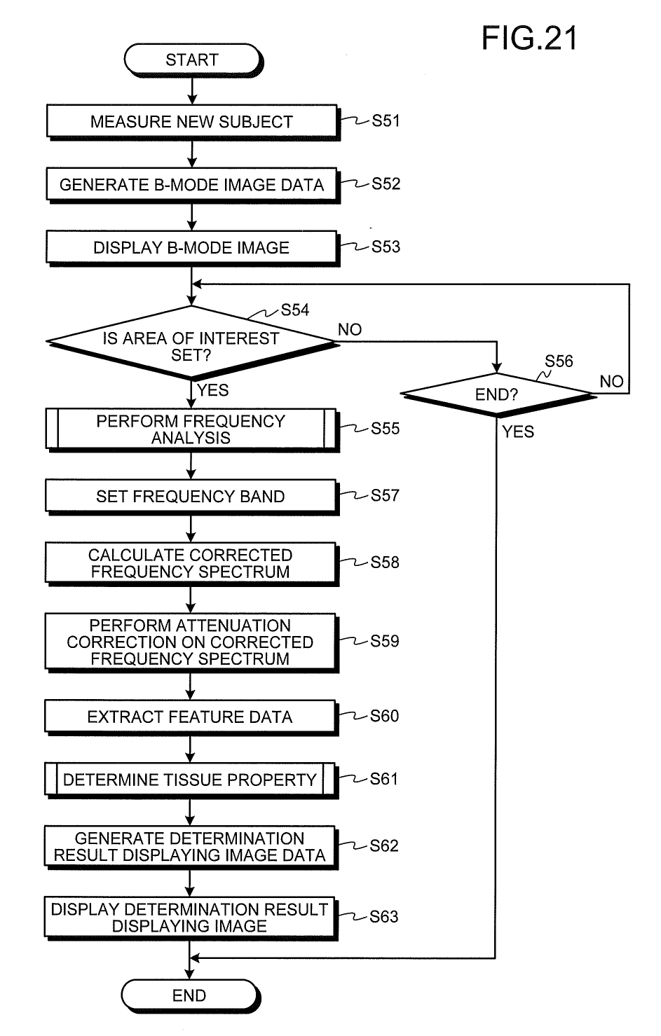

FIG. 21 is a flowchart of an outline of an attenuation correcting process performed by the ultrasonic observation apparatus according to the second embodiment. In FIG. 21, processes of steps S51 to S58 sequentially correspond to processes of steps S1 to S8 in FIG. 4.

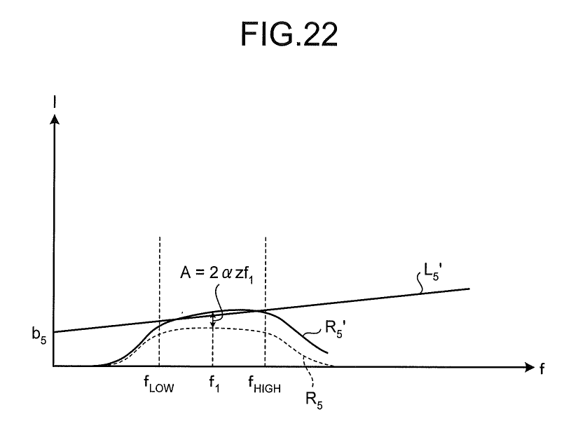

At step S59, the attenuation corrector 442 performs the attenuation correction on the corrected frequency spectrum calculated by the corrected frequency spectrum calculator 43 (step S59). FIG. 22 schematically shows an outline of the process at step S59. As shown in FIG. 22, the attenuation corrector 442 obtains a new frequency spectrum curve R₅′ by performing, with respect to a corrected frequency spectrum curve R₅, a correction in which an attenuation amount A in expression (1) explained above is added to the intensity I on all the frequencies f. Thus, it is possible to obtain a frequency spectrum in which the contribution of the attenuation associated with the transmission of ultrasonic waves is reduced.

After this, the approximating unit 441 extracts feature data of frequency spectrum via the regression analysis on all the frequency spectra on which the attenuation correction is performed by the attenuation corrector 442 (step S60). Specifically, the approximating unit 441 calculates the slope a, the intercept b, and the intensity c in the middle frequency f_{MID} of the primary expression via the regression analysis. A straight line L₅′ shown in FIG. 22 is a regression line (intercept b₅) obtained by performing the feature data extracting process on the corrected frequency spectrum curve R₅ at step S60.

Processes of steps S61 to S63 sequentially correspond to the processes of steps S11 to S13 in FIG. 4.

According to the second embodiment of the present invention explained so far, it is possible to clearly determine the difference in tissues without using an amount of strain and a modulus of elasticity of biological tissues since a frequency spectrum is calculated by analyzing a frequency of received ultrasonic waves, a frequency band used in approximating the frequency spectrum is set, the frequency spectrum is corrected based on a reference spectrum read out from the storage unit that stores the reference spectrum obtained based on a frequency of ultrasonic waves received from the reference reflector, the attenuation correcting process in which the contribution of ultrasonic attenuation which depends on the reception depth and the frequency of ultrasonic waves is reduced is performed on the corrected frequency spectrum, and then feature data of the subject is extracted by performing the approximating process. Hence, it is possible to enable distinguishing a tissue property accurately and to enhance the reliability for the observation result.

According to the second embodiment, it is possible to remove an influence of the attenuation associated with the transmission of ultrasonic waves and to perform a tissue property determination with higher accuracy since the attenuation correction is performed on the corrected frequency spectrum.

According to the second embodiment, it is possible to remove an influence of the attenuation associated with the transmission of ultrasonic waves and to perform a tissue property determination with even higher accuracy since the frequency band is determined so that the band width becomes narrower and the maximum frequency becomes smaller as the reception depth is larger.

### Third Embodiment

A third embodiment of the present invention differs from the first embodiment in the tissue property determining process by the tissue property determining unit. A configuration of an ultrasonic observation apparatus according to the third embodiment is the same as that of the ultrasonic observation apparatus 1 explained in the first embodiment. Therefore, an identical component corresponding to a component of the ultrasonic observation apparatus 1 will be assigned with the same reference symbol in the explanation below.

The tissue property determining unit 45, after making up new populations by adding feature data (a, b, c) to each of the groups G_{μ}, G_{ν}, and G_{ρ} (see FIG. 18) respectively for the tissue properties μ, ν, and ρ, obtains a standard deviation for each kind, constituting each tissue property, of the feature data.

After that, the tissue property determining unit 45 calculates a difference (hereinafter referred to simply as “difference in standard deviation”) between a standard deviation of each kind of the feature data of the groups G_{μ}, G_{ν}, and G_{ρ} in original populations constituted only by known subjects and a standard deviation of each kind of the feature data of the groups G_{μ}, G_{ν}, and G_{ρ} in the new populations in which the new subject is added each, and determines that a tissue property corresponding to a group including feature data whose difference in standard deviation is the smallest should be the tissue property of the subject.

Here, the tissue property determining unit 45 may calculate the difference in standard deviation only with respect to the difference in standard deviation of given pieces of feature data selected in advance among plural pieces of feature data. The selection of the feature data in this case may be arbitrarily performed by the operator or may be automatically performed by the ultrasonic observation apparatus 1.

Besides, the tissue property determining unit 45 may calculate a value in which a weight is arbitrarily added to the difference in standard deviation of all the feature data in each group and determine that a tissue property corresponding to a group the calculated value of which is the smallest should be the tissue property of the subject. In this case, when the feature data is “slope a, intercept b, and intensity c”, for example, the tissue property determining unit 45 performs, by setting weights for the slop a, the intercept b, and the intensity c, respectively to wₐ, w_{b} and w_{c}, a calculation of wₐ·(difference in standard deviation of a)+w_{b}·(difference in standard deviation of b)+w_{c}·(difference in standard deviation of c), and determines the tissue property of the subject based on the calculated value. Here, the values for the weights wₐ, w_{b} and w_{c} may be arbitrarily set by the operator or may be automatically set by the ultrasonic observation apparatus 1.

Besides, the tissue property determining unit 45 may calculate the square root of a value in which a weight is arbitrarily added to the square of the difference in standard deviation of all the feature data for each group, and determine that a tissue property corresponding to a group the square root of which is the smallest should be the tissue property of the subject. In this case, when the feature data is “slop a, intercept b, and intensity c”, for example, the tissue property determining unit 45 performs, by setting weights for the slop a, the intercept b, and the intensity c, respectively to w′ₐ, w′_{b} and w′_{c}, a calculation of {w'ₐ·(difference in standard deviation of a)²+w′_{b}·(difference in standard deviation of b)²⁺ w′_{c}·(difference in standard deviation of c)²}^{1/2}, and makes the tissue property determination based on the calculated value. Here in this case, too, the values for the weights w′ₐ, w′_{b} and w′_{c} may be arbitrarily set by the operator or may be automatically set by the ultrasonic observation apparatus 1.

According to the third embodiment of the present invention explained so far, it is possible to enable distinguishing a tissue property accurately, to enhance the reliability for the observation result, and to perform a tissue property determination with higher accuracy by removing an influence of the attenuation associated with the transmission of ultrasonic waves, similarly to the first embodiment explained above.

While the tissue property determining unit 45 determines the tissue property based on the change in standard deviation of each kind of feature data between an original population and another population in which a new subject is added in the third embodiment, this configuration is just one example. The tissue property determining unit 45 may determine the tissue property based on a change in average of each kind of the feature data between the original population and another population in which a new subject is added, for example.

While the embodiments of the present invention are explained so far, the present invention is not limited only to the first to the third embodiments explained above. In other words, the present invention may cover various embodiments within a scope not departing from the technical ideas as defined by the appended claims.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic observation apparatus that transmits an ultrasonic wave to a subject and receives an ultrasonic wave reflected by the subject, the ultrasonic observation apparatus comprising:
a reference spectrum storage unit that stores a first reference spectrum in a first reception depth range and a second reference spectrum in a second reception depth range obtained based on a frequency of an ultrasonic wave received from a reference reflector;
a frequency analyzer that calculates a frequency spectrum by analyzing a frequency of the received ultrasonic wave; and
a corrected frequency spectrum calculator that calculates a corrected frequency spectrum by determining whether a reception depth of the frequency spectrum calculated by the frequency analyzer is the first reception depth range or the second reception depth range, and obtaining a difference, in a case of the first reception depth range, between the first reference spectrum and the frequency spectrum and a difference, in a case of the second reception depth range, between the second reference spectrum and the frequency spectrum.

2. The ultrasonic observation apparatus according to claim 1, comprising
a feature data extracting unit that extracts feature data of the subject by performing, on the corrected frequency spectrum calculated by the corrected frequency spectrum calculator, an approximating process and an attenuation correcting process in which a contribution of an attenuation which arises in a transmission of an ultrasonic wave depending on a reception depth and a frequency of the ultrasonic wave is reduced.

3. The ultrasonic observation apparatus according to claim 2, wherein the feature data extracting unit includes:
an approximating unit that extracts before-correction feature data before the attenuation correcting process is performed by performing the approximating process on the frequency spectrum calculated by the frequency analyzer; and
an attenuation corrector that extracts the feature data of the frequency spectrum by performing the attenuation correcting process on the before-correction feature data extracted by the approximating unit.

4. The ultrasonic observation apparatus according to claim 2, wherein the feature data extracting unit includes:
an attenuation corrector that performs the attenuation correcting process on the frequency spectrum; and
an approximating unit that extracts the feature data of the frequency spectrum by performing the attenuation correcting process on the frequency spectrum corrected by the attenuation corrector.

5. The ultrasonic observation apparatus according to claim 3, wherein the attenuation corrector performs a larger correction as a reception depth of the ultrasonic wave is larger.

6. The ultrasonic observation apparatus according to claim 3, wherein the approximating unit approximates the frequency spectrum by a polynomial expression via a regression analysis.

7. The ultrasonic observation apparatus according to claim 6, wherein the approximating unit approximates the frequency spectrum by a primary expression and extracts plural kinds of feature data including at least two of a slope of the primary expression, an intercept of the primary expression, and an intensity which is defined by using the slope, the intercept, and a specific frequency included in a frequency range of the frequency spectrum.

8. The ultrasonic observation apparatus according to claim 2, further comprising:
a storage unit that stores, by associating with tissue properties of a plurality of known subjects, feature data of frequency spectra extracted based on ultrasonic waves reflected by the plurality of respective known subjects; and
a tissue property determining unit that determines a tissue property in a predetermined area of the subject by using the feature data stored by being associated with the plurality of known subjects by the storage unit and the feature data extracted by the feature data extracting unit.

9. The ultrasonic observation apparatus according to claim 8, wherein
the storage unit stores an average of each kind of feature data in groups classified for each tissue property of the plurality of known subjects, and
the tissue property determining unit sets a feature data space whose component is at least one of the plural kinds of feature data, and determines the tissue property of the subject based on a distance, on the feature data space, between a subject point having, as a coordinate in the feature data space, the feature data which is the component of the feature data space among the feature data of the frequency spectrum of the subject and a known subject average point having, as a coordinate in the feature data space, an average of the feature data which is the component of the feature data space among respective kinds of the feature data in the groups of the plurality of known subjects.

10. The ultrasonic observation apparatus according to claim 8, wherein the tissue property determining unit calculates a standard deviation of feature data in populations, which are formed by adding the feature data of the subject in groups classified for each tissue property of the plurality of known subjects, and determines that a tissue property corresponding to a group having feature data whose difference between the standard deviation and a standard deviation of feature data in the groups is smallest should be the tissue property of the subject.

11. The ultrasonic observation apparatus according to claim 1, comprising
a display unit that generates visual information corresponding to feature data of the subject and displays image generated based on the generated visual information and the received ultrasonic wave.

12. The ultrasonic observation apparatus according to claim 11, wherein the visual information is a variable constituting a color space.

13. The ultrasonic observation apparatus according to claim 3, wherein the attenuation corrector performs the attenuation correcting process by using a predetermined attenuation rate which is determined depending on the subject.

14. The ultrasonic observation apparatus according to claim 13, wherein the attenuation corrector further includes an attenuation rate setting unit that sets the predetermined attenuation rate which is determined depending on the subject.

15. An operation method of an ultrasonic observation apparatus that transmits an ultrasonic wave to a subject and receives an ultrasonic wave reflected by the subject, the operation method comprising:
calculating a frequency spectrum by a frequency analyzer by analyzing a frequency of a received ultrasonic wave;
storing a first reference spectrum in a first reception depth range and a second reference spectrum in a second reception depth range obtained based on a frequency of an ultrasonic wave received from a reference reflector; and
calculating a corrected frequency spectrum by determining whether a reception depth of the frequency spectrum calculated at the calculating is the first reception depth range or the second reception depth range and by obtaining a difference, in a case of the first reception depth range, between the first reference spectrum and the frequency spectrum and a difference, in a case of the second reception depth range, between the second reference spectrum and the frequency spectrum.

16. A non-transitory computer readable recording medium with an executable program stored thereon, wherein the program instructs a processor to execute:
calculating a frequency spectrum by a frequency analyzer by analyzing a frequency of a received ultrasonic wave;
storing a first reference spectrum in a first reception depth range and a second reference spectrum in a second reception depth range obtained based on a frequency of an ultrasonic wave received from a reference reflector; and
calculating a corrected frequency spectrum by determining whether a reception depth of the frequency spectrum calculated at the calculating is the first reception depth range or the second reception depth range and by obtaining a difference, in a case of the first reception depth range, between the first reference spectrum and the frequency spectrum and a difference, in a case of the second reception depth range, between the second reference spectrum and the frequency spectrum.
